(19) United States
(12) Patent Application Publication
(10) Pub. No.: US 2023/0316784 A1
(43) Pub. Date: Oct. 5, 2023

(54) METHOD FOR A VEHICLE FOR DETERMINING AN INJURY INFORMATION AND VEHICLE ELECTRONIC CONTROL DEVICE FOR DETERMINING AN INJURY INFORMATION

(71) Applicant: CONTINENTAL AUTOMOTIVE GMBH, Hannover (DE)

(72) Inventor: Suchitra Damodharan, Electronic City (IN)

(73) Assignee: Continental Automotive Technologies GmbH, Hannover (DE)

(21) Appl. No.: 18/043,289

(22) PCT Filed: Dec. 4, 2020
(86) PCT No.: PCT/EP2020/084621

§ 371 Date: Feb. 27, 2023

(30) Foreign Application Priority Data
Aug. 31, 2020 (IN) 202041037440

(51) Int. Cl. G06V 20/59; G16H 50/30; B60W 40/08

(52) U.S. Cl. CPC G06V 20/597; G16H 50/30; B60W 40/08; B60W 2040/0881; B60W 2540/221; B60W 2540/26

(57) ABSTRACT

A vehicle and method for determining an injury information of a vehicle occupant after an accident of the vehicle based on sensing, by a vital sensor, at least one physiological signal of the occupant and providing a first output signal indicative of an internal trauma of the occupant to the vehicle electronic control device; providing to the vehicle electronic control device at least one second output signal from a vehicle internal sensor indicative of an external physical injury of the occupant; and determining, by the vehicle electronic control device, an emergency severity index indicative of a life-criticality status based on the first output signal and the second output signal.

### CROSS-REFERENCE TO RELATED APPLICATIONS

The present application is a National Stage Application under 35 U.S.C. § 371 of International Patent Application No.PCT/EP2020/084621 filed on Dec. 4, 2020, and claims priority from Indian Patent Application No. 202041037440 filed on Aug. 31, 2020, in the Indian Patent and Trademark Office, the disclosures of which are herein incorporated by reference in their entireties.

### BACKGROUND

### 1. Field

Embodiments of the present application relate to a method for a vehicle for determining an injury information about an injury to a vehicle occupant after an accident. Embodiments of the present application also relate to a vehicle electronic control device for determining an injury information about an injury to a vehicle occupant after an accident.

### 2. Description of the Related Art

The safety of occupants in vehicles assumes an important role in the development of vehicles. Safety approaches frequently include several measures for increasing driving safety, from a structural protection of the vehicle cabin, to occupant restraint systems like airbags and belt tightening systems, to an automated transmission of emergency calls with the aim of being able to better protect occupants in the vehicle in case of an accident.

Automatic emergency call methods and systems are well known, wherein in the event of a vehicular accident an emergency call is transmitted particularly to a rescue control center or the like provided with information related to the accident. Such systems are also known as eCall systems. This is intended to ensure that a rescue chain is set up as quickly as possible. To this end, usually information on the position and direction of travel of the vehicle involved in the accident is provided with the emergency call.

For the survival and the maximum achievable rehabilitation success of an injured occupant, the length of time from the accident to the first effective assistance and thus to the arrival of the professional rescue service and the quality of the initial treatment at the accident location as well as the subsequent clinical care are of decisive importance in emergency care. With the known emergency call methods and systems, a noticeable time saving can be achieved in the rescue phase after an accident of the vehicle by shortened search times or a shortened approach time. However, with the known emergency call methods and systems it can be regarded as disadvantageous that a disposition decision for rescue services to be deployed is left to the rescue control center and that no detailed information regarding crash and injury severity is provided.

### SUMMARY

In view of the foregoing, an aspect of an embodiment is to provide a method and a vehicle electronic control device for determining an injury information of a vehicle occupant after an accident of the vehicle, which ensure an accurate and proper determination of the injury information and thus enable a quick and efficient post-crash care and thus an enhanced safety of a vehicle occupant.

According to one aspect of an embodiment, a method for determining an injury information of a vehicle occupant after an accident of the vehicle is provided. The method comprising:
sensing, by a vital sensor, at least one physiological signal of the occupant and providing a first output signal indicative of an internal trauma of the occupant to the vehicle electronic control device;
providing to the vehicle electronic control device at least one second output signal from a vehicle internal sensor indicative of an external physical injury of the occupant; and
determining, by the vehicle electronic control device, an emergency severity index indicative of a life-criticality status based on the first output signal and the second output signal.

The embodiment is based on the consideration that a quick and efficient post-crash care of an injured occupant can be achieved if an injury information of the occupant is determined, and wherein therefore an emergency severity index indicative of a life-criticality status is determined. The embodiment is further based on the consideration that and accurate and confident determination can be achieved if the determination is based also on post-crash data, and wherein the determination is based on a combination of physiological data or internal trauma data of the occupant and external physical injury data of the occupant.

In accordance with an aspect of an embodiment, an accurate and proper determination of an injury information is enabled and thus a quick and efficient post-crash care and thus an enhanced safety of a vehicle occupant are ensured.

The steps of the method are preferably done in a post-crash phase, particularly immediately after the occurring of the crash or accident.

The external physical injury can be particularly an external bleeding of the occupant and/or a penetrateable injury of the occupant.

The method can use more than one vital sensor and preferably more than one physiological signal is sensed, particularly various physiological signals. Preferably, the one or more vital sensors are part of an occupant monitoring system that is capable of acquiring, particularly various, physiological signals. Furthermore, the method can use more than one vehicle internal sensor.

The vehicle internal sensor is an interior sensor situated in the vehicle, wherein the vehicle internal sensor particularly is integrated in the roof lining between the vehicle front seats and/or in an area close to the rear-view mirror of the vehicle.

Preferably, in a pre-crash phase a dormant stage of the vital sensor and the vehicle internal sensor is activated to prevent damage of the sensors due to the following crash or accident. After the crash or accident, i.e. in a post-crash phase, the respective sensors are re-activated. Thereby, a power reserve can be utilized to re-activate the sensors.

In case of an accident of the vehicle with more than one occupant within the vehicle, for each of the occupants an emergency severity index is determined.

Furthermore, in case of an accident of the vehicle with more than one occupant within the vehicle, it is preferably prioritized based on the age of the occupants. Accordingly, particularly children and elderly are prioritized when performing the method. Regarding the step of sensing, by a vital sensor, at least one physiological signal of the occupant and providing a first output signal indicative of an internal trauma of the occupant to the vehicle electronic control device, alternatively or additionally to the age of the occupants it is prioritized based on medical ailments in history of the occupants which can be retrieved from a storage unit of the vehicle electronic control device. Accordingly, particularly children and elderly and/or occupants with medical ailments in history are prioritized when performing this step. Regarding the step of providing to the vehicle electronic control device at least one second output signal from a vehicle internal sensor indicative of an external physical injury of the occupant, alternatively or additionally to the age of the occupants it is prioritized based on a status of a restraint means for the occupants. Accordingly, particularly children and elderly and/or occupants whose corresponding restraint means are ineffective or disabled are prioritized when performing this step.

According to an advantageous specific embodiment, the at least one physiological signal is at least one of respiration rate, heartbeat, heart rate, pulse rate, body temperature, oxygen saturation or brain wave.

According to another specific embodiment, the vehicle internal sensor is designed as an optical sensor, preferably as an imaging sensor, for example an interior camera. Particularly, the vehicle internal sensor is designed as a thermal imaging sensor, wherein by using a thermal imaging sensor also an internal injury of the occupant can be additionally be detected.

In case of an external bleeding of the occupant as an external physical injury particularly a video processing of blood detection on contact surfaces and the occupant can be used to derive the at least one second output signal.

Preferably in case of a penetrateable injury of the occupant the at least one second output signal is derived based on occupant state information in the pre-crash phase.

According to another specific embodiment, the method further comprising:
sensing, by a crash sensor, at least one crash-related data and providing a third output signal indicative of a crash dynamic to the vehicle electronic control device;
determining at least one of an impact location or a damage extend based on the third output signal and generating, by the vehicle electronic control device, a fourth output signal indicative of the impact location and/or the damage extend; and
determining, by the vehicle electronic control device, the emergency severity index indicative of a life-criticality status based on the first output signal, the second output signal and the fourth output signal.

In this way, the determination of the emergency severity index further takes into account the impact location and/or a damage extend derived from a signal of a crash sensor. The determination of an injury information will be further precisionized in this was.

The step of sensing, by a crash sensor, at least one crash-related data and providing a third output signal indicative of a crash dynamic to the vehicle electronic control device is preferably done in the pre-crash phase and/or at the point when the crash or accident occurs, i.e. whereas the crash-phase.

In the step of determining at least one of an impact location or a damage extend only the impact location can be determined. Alternatively, only the damage extend can be determined. It is also alternatively possible that in this step an impact location and a damage extend are determined. In case of determining an impact location, the information of the one or more impact locations can also be used for the vehicle internal sensor to specify the regions of scanning. Furthermore, the determined one or more impact locations can be categorized based on the level of possibility of being an impact location. Different categories like ‘High (Certain)’, Medium (Probable)’ and ‘Low (Improbable)’ can be used for this and can be assigned to the respective impact location.

Preferably, more than one crash sensor is used, and more than one crash-related data is sensed, particularly various crash-related data. The crash sensor can particularly be designed as an acceleration sensor, an angular rate sensor, a pressure sensor or a surrounding sensor like a radar sensor, a lidar sensor or an exterior camera.

In a preferred further embodiment, the at least one crash-related data is at least one of a vehicle acceleration, a collision velocity, a collision angle, or a pressure. Combinations of these data are also possible.

In a preferred further embodiment, the determining of at least one of the impact location or the damage extend is further based on at least one of the following information: a seat position of the occupant, a status of an occupant restraint system, presence of a mobile object within the vehicle cabin, or a design of the vehicle cabin. This information can preferably be determined already in a phase before the crash or accident.

According to another specific embodiment, the method further comprising:
generating, by the vehicle electronic control device, a control signal for triggering a transmitting of an emergency call in response to the determined emergency severity index.

In a preferred further embodiment, the emergency call comprises at least one of the following emergency calls: emergency call for immediate assistance from nearby vehicle, emergency call for automatic collision notification system, emergency call for emergency medical service personnel, or emergency call to a personal contact notification of the occupant. So, in dependence of the determined emergency severity index an emergency call to a specific recipient is transmitted.

In a preferred further embodiment, the determined emergency severity index is provided with the emergency call. Thus, with the emergency call also the emergency severity index is transmitted and provided to the recipient.

In a preferred further embodiment, at least on of an occupant seat position or an occupant age is provided with the emergency call. Thus, with the emergency call also the occupant seat position and/or the occupant age is transmitted and provided to the recipient. Furthermore, also the number of occupants in the accident vehicle and the number of children or elderly, which can be derived particularly from the age of the occupant, can be provided with the emergency call.

According to another specific embodiment, the emergency severity index is stored in a storage unit of the vehicle electronic control device. In this way, necessary information is available for emergency response personnel at the location of the accident.

According to another aspect of an embodiment, a vehicle electronic control device for determining an injury information about an injury to a vehicle occupant after an accident is provided, wherein the vehicle electronic control device is configured to:
obtain a first output signal indicative of an internal trauma of the occupant from a vital sensor sensing at least one physiological signal of the occupant;
obtain at least one second output signal from a vehicle internal sensor indicative of an external physical injury of the occupant; and
determine an emergency severity index indicative of a life-criticality status based on the first output signal and the second output signal.

The advantages and the specific and preferred embodiments described for the method also apply accordingly to the vehicle electronic control device according to an embodiment.

### BRIEF DESCRIPTION OF THE DRAWINGS

The drawings described herein are used to provide a further understanding for the present application, and constitute a part of the present application. The exemplary embodiments and descriptions are used to explain the present application, and do not constitute an improper limitation to the present application. In the drawings:

FIG. 1 shows a flow chart of a method for determining an injury information of a vehicle occupant after an accident of the vehicle according to a preferred embodiment; and

FIG. 2 shows a flow chart of a method for determining the emergency severity index according to FIG. 1.

### DETAILED DESCRIPTION

The following describes the present application in detail with reference to the accompanying drawings and in combination with embodiments. It should be noted that, without conflicts, the embodiments and features in the embodiments may be combined with each other. Parts corresponding to each other are always provided with the same reference signs in all figures.

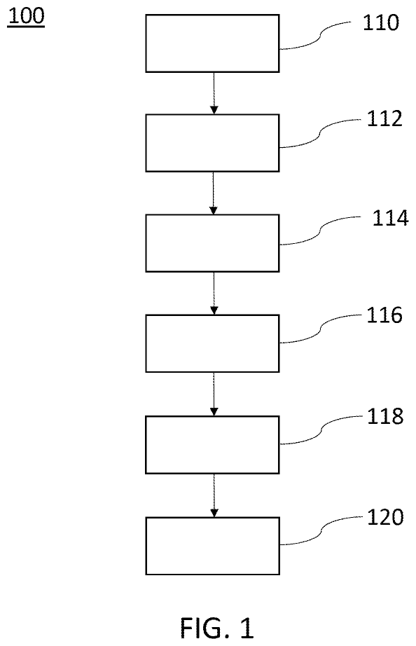

FIG. 1 shows a flow chart of a method 100 for determining an injury information of a vehicle occupant after an accident of the vehicle.

In step 110 crash-related data is sensed by a crash sensor whereas the crash or accident of the vehicle and a third output signal indicative of a crash dynamic is provided to a vehicle electronic control device which is designed as an airbag control device.

In step 112 an impact location and a damage extend are determined based on the third output signal and on a seat position of the occupant, wherein the seat position of the occupant was determined already in a phase before the crash or accident. A fourth output signal indicative of the impact location and the damage extend is then generated by the vehicle electronic control device.

In step 114, after occurring of the accident, several physiological signals of the occupant are sensed by different vital sensors, wherein these vital sensors are part of an occupant monitoring system, and a first output signal indicative of an internal trauma of the occupant is provided to the vehicle electronic control device.

In step 116 a second output signal from a vehicle internal sensor, designed as an interior camera, indicative of an external physical injury of the occupant like a bleeding is provided to the vehicle electronic control device.

In step 118 an emergency severity index indicative of a life-criticality status is determined by the vehicle electronic control device based on the first output signal, the second output signal and the fourth output signal.

In this way, an accurate and confident determination of a life-criticality status of the occupant can be achieved.

In case of an accident of the vehicle with more than one occupant within the vehicle, for each of the occupants an emergency severity index is determined by the described steps.

In step 120 a control signal for triggering a transmitting of an emergency call in response to the determined emergency severity index is generated by the vehicle electronic control device and the emergency call is transmitted to a respective recipient. With the emergency call the position of the vehicle, the number of occupants of the vehicle, the number of children or elderly among the occupants, the seat position of the occupant and the determined emergency severity index for the occupant is provided or transmitted. In this step the provided or transmitted information is also stored in a storage unit of the vehicle electronic control device.

In this way, a quick and efficient post-crash care of an injured occupant can be achieved based on the accurate and confident determination of the emergency severity index indicative of the life-criticality status of the occupant.

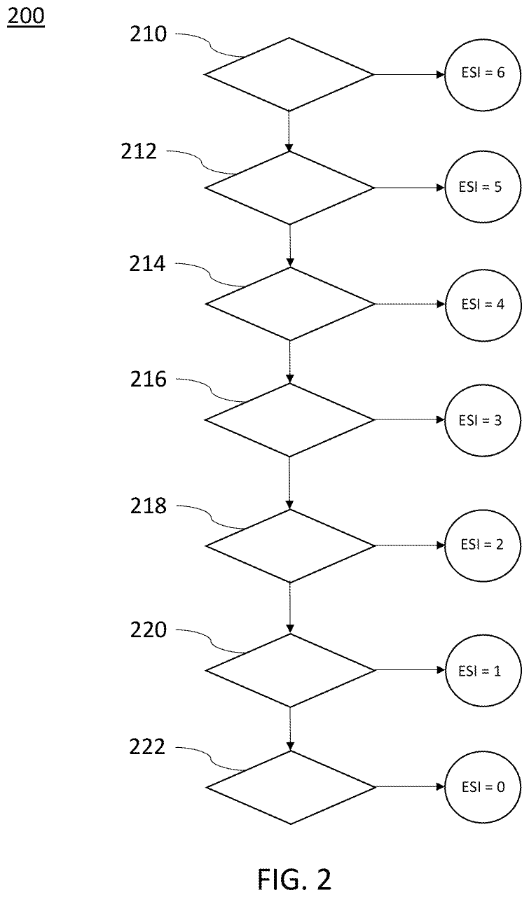

FIG. 2 shows a flow chart of a method 200 for determining the emergency severity index according to FIG. 1.

In step 210 it is determined if the occupant is alive. This determination is done based on the sensed physiological signals or the first output signal. If none vital can be sensed or detected, then the value 6 is assigned as the emergency severity index (ESI).

If at least one of the vitals is being sensed or detected, then in step 212 it is determined if the occupant is dying. This determination is done based on the sensed physiological signals respiration rate and heart rate or the respective first output signal. If these vitals fading, i.e. reach critical levels, then the value 5 is assigned as the emergency severity index (ESI).

If these vitals do not fade, i.e. do not reach critical levels, then in step 214 the criticality of the occupant vitals is determined. This determination is done based on the sensed physiological signals respiration rate, heart rate and oxygen saturation or the respective first output signal. If these vitals are below an acceptable predetermined vital threshold, then the value 4 is assigned as the emergency severity index (ESI).

If these vitals are not below the predetermined vital threshold, then in step 216 the neurological state of consciousness of the occupant is determined according to the known Glasgow Coma Scale based on the sensed physiological signals or the first output signal. If the determined Glasgow Coma Scale is below a predetermined limit value, then the value 3 is assigned as the emergency severity index (ESI).

If the determined Glasgow Coma Scale is above the predetermined limit value, then in step 218 an occupant injury score is determined based on the number of physical injuries and their severity provided by the second output signal. If the determined occupant injury score is above a predetermined injury threshold, then the value 2 is assigned as the emergency severity index (ESI).

If the determined occupant injury score is below the predetermined injury threshold, then in step 220 an occupant injury is estimated based on a probable impact analysis and an injury estimate which is based on the fourth output signal. If an injury to the occupant is assumed, then the value 1 is assigned as the emergency severity index (ESI).

If no injury to the occupant is assumed, then in step 222 it is ascertained that no medical assistance is needed and the value 0 is assigned as the emergency severity index (ESI).

In the event that all of the information, data or signals are not accessible, then the value 9 is assigned as the emergency severity index (ESI).

1. A method of a vehicle determining an injury information of a vehicle occupant after an accident of the vehicle, the method comprising:
sensing, by a vital sensor, at least one physiological signal of the occupant and providing a first output signal indicative of an internal trauma of the occupant to the vehicle electronic control device;
providing to the vehicle electronic control device at least one second output signal from a vehicle internal sensor indicative of an external physical injury of the occupant; and
determining, by the vehicle electronic control device, an emergency severity index indicative of a life-criticality status based on the first output signal and the second output signal.

2. The method according to claim 1, wherein the at least one physiological signal is at least one of respiration rate, heartbeat, heart rate, pulse rate, body temperature, oxygen saturation or brain wave.

3. The method according to claim 2, wherein the vehicle internal sensor is designed as an optical sensor.

4. The method according to claim 1, further comprising:
sensing, by a crash sensor, at least one crash-related data and providing a third output signal indicative of a crash dynamic to the vehicle electronic control device;
determining at least one of an impact location or a damage extend based on the third output signal and generating, by the vehicle electronic control device, a fourth output signal indicative of the impact location and/or the damage extend; and
determining, by the vehicle electronic control device, the emergency severity index indicative of a life-criticality status based on the first output signal, the second output signal and the fourth output signal.

5. The method according to claim 4, wherein the at least one crash-related data is at least one of a vehicle acceleration, a collision velocity, a collision angle, or a pressure.

6. The method according to claim 5, wherein the determining of at least one of the impact location or the damage extend is further based on at least one of the following information: a seat position of the occupant, a status of an occupant restraint system, presence of a mobile object within the vehicle cabin, or a design of the vehicle cabin.

7. The method according to claim 6, further comprising:
generating, by the vehicle electronic control device, a control signal for triggering a transmitting of an emergency call in response to the determined emergency severity index.

8. The method according to claim 7, wherein the emergency call comprises at least one of the following emergency calls:
emergency call for immediate assistance from nearby vehicle, emergency call for automatic collision notification system, emergency call for emergency medical service personnel, or emergency call to a personal contact notification of the occupant.

9. The method according to claim 8, wherein the determined emergency severity index is provided with the emergency call.

10. The method according to claim 9, wherein at least on of an occupant seat position or an occupant age is provided with the emergency call.

11. The method according to claim 10, wherein the emergency severity index is stored in a storage unit of the vehicle electronic control device.

12. (canceled)
